(19) United States
(12) Patent Application Publication
(10) Pub. No.: US 2003/0012829 A1
(43) Pub. Date: Jan. 16, 2003

(54) Cold concentration of plant extract

(76) Inventor: Dennis Itami, Nampa, ID (US)

Correspondence Address: PEDERSEN & COMPANY, PLLC, P.O. BOX 2666, BOISE, ID, 83701, US

(21) Appl. No.: 10/164,302

(22) Filed: Jun. 4, 2002

Related U.S. Application Data
(60) Provisional application No. 60/295,972, filed on Jun. 4, 2001.

(51) Int. Cl. A61K 35/78; A23L 2/00

(52) U.S. Cl. 424/725; 426/599

(57) ABSTRACT

An economical and efficient apparatus and method for concentration of plant extracts is disclosed. The apparatus is in one embodiment a field harvester in communication with a plant cutter and collector which delivers the plant material to a macerator carried by the harvester. From the macerated material, liquid plant values, including enzymes, etc., are selectively obtained by means of, for example, an on-board screen press and/or solvent extractor. Then, the liquid plant values are immediately actively cooled to a temperature substantially less than ambient. Then, while still cooled, the liquid values and further concentrated by centrifuging, decanting, filtering, etc., without substantially increasing the temperature of the plant values. During the concentration steps, or preferably continuously during the handling process of the liquid plant values, they are quickly chilled to a temperature substantially less than ambient in order to stabilize them and preserve them against degradation.

### DESCRIPTION

This application claims priority from U.S. Provisional Patent Application Serial No. 60/295,972, filed Jun. 4, 2001, hereby incorporated herein.

### BACKGROUND OF THE INVENTION

1. Field of the Invention

This invention relates generally to food and food supplement processing. More specifically, this invention relates to a method of cold concentration of plant extract for use as a food supplement.

2. Related Art

U.S. Pat. No. 4,287,708 (Neel Jr.) discloses a field harvester which mows, gathers and grinds alfalfa. The protein-rich liquid extract of the ground alfalfa is collected and refrigerated, and the moisture content of the cut and ground fiber is controlled for pelletizing.

U.S. Pat. No. 5,820,916 (Sagliano, et al.) discloses a method for preserving nutrients in wheatgrass juice. The juice is obtained from extractors which will not allow the juice temperature to exceed 90° F. (32° C.). After juicing, the wheatgrass juice is immediately freeze-dried.

Still, there is a need for an efficient and economical method for cold concentration of liquid plant extracts. This need is acute for those many liquid plant extract products which are easily damaged by heating, or even by ambient temperature. Also, this need is acute for those liquid extracts which are obtained in large volumes, so that economy and efficiency dictate, at least in part, the methods and equipment for the concentration thereof. The present invention addresses this need.

### SUMMARY OF THE INVENTION

The present invention is in one embodiment an economical and efficient apparatus and method for on-board harvester cold concentration of plant extracts. The apparatus is a field harvester in communication with a plant cutter and collector which delivers the plant material to a macerator carried by the harvester. From the macerated material, liquid plant values, including enzymes, etc., are selectively obtained by means of, for example, an on-board screen press and/or solvent extractor. Solid plant values, including fiber, etc., are separated and preferably stored temporarily on-board or otherwise handled off-board.

Then, in this embodiment the liquid plant values are further concentrated on-board by any combination of centrifuging, decanting, filtering, etc., without substantially increasing the temperature of the plant values. In fact, preferably the temperature of the liquid plant values are actively reduced. During the further concentration steps, or preferably continuously during the on-board handling process of the liquid plant values, they are quickly chilled to a temperature substantially less than ambient in order to stabilize them and preserve them against degradation. Finally, the chilled plant extract concentrate is unloaded into low-temperature-controlled storage at a transfer station or onto another tank-carrying vehicle for further handling.

In another embodiment of the present invention, the chilled plant extract from the field, with or without prior, in-field concentration, is further processed while continuously maintained at reduced temperature, either to a chilled or frozen concentrate, or up to the point of spray drying the cold concentrate into a powder. Further processing may, but need not, include centrifuging, decanting, filtering, reverse osmosis, etc. In any event, the further processing is done at a temperature substantially less than ambient. This way, the desirable food values in the plant extract may be further preserved and maximized.

### BRIEF DESCRIPTION OF THE DRAWINGS

FIG. 1 is a schematic process flow diagram of one embodiment of the cold concentration method of the present invention.

FIG. 2 is a schematic side view of one embodiment of the field harvester/concentrator apparatus of the present invention.

FIG. 3 is a side schematic view of another embodiment of the present invention, wherein a chilled juice concentrate from the field is further processed at temperatures substantially less than ambient.

FIG. 4 is a schematic temperature—time chart of a cold concentration process according to the present invention.

### DESCRIPTION OF THE PREFERRED EMBODIMENTS

Referring to the Figures, there are depicted several, but not all, embodiments of the present invention.

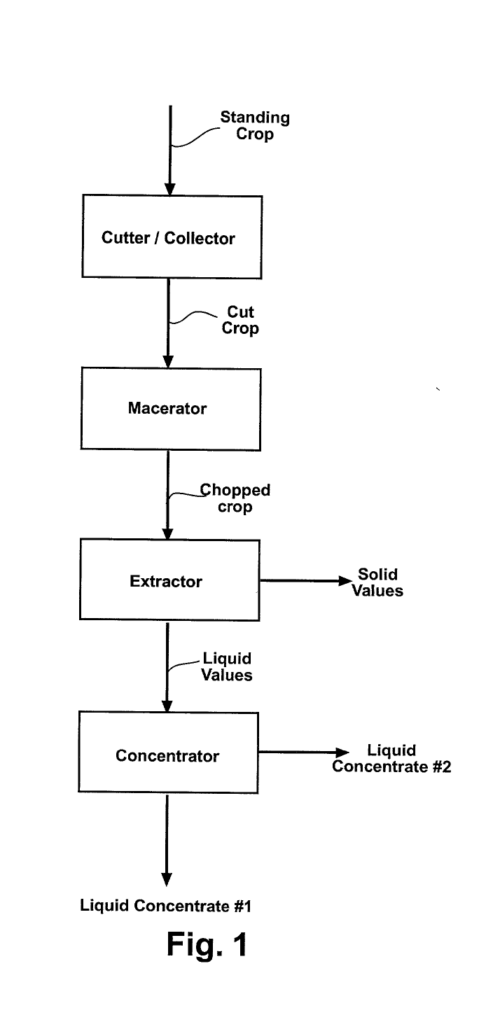

FIG. 1 depicts a schematic process flow diagram of one embodiment of the cold concentration method of the present invention. According to FIG. 1, a standing crop is cut and collected, preferably by a harvester apparatus of the present invention. Alternatively, a previously cut crop which has been left temporarily in the field may be gathered up off the ground. Typical mowers and cutter heads, similar to those for grain combines, will suffice. Also, typical conveyors, whether moving belts or fluted rollers/augers, will also suffice.

Then, the cut and gathered crop is macerated, or ground into fine pieces. Preferably, this step is accomplished with a suitable hammer mill, but other, alternative conventional choppers/grinders will also suffice.

After maceration, the chopped crop is subjected to extraction in order to separate liquid from solid plant values. Preferably, the extraction is accomplished, at least in part, with a rotary screen press. This way, liquid values pass through the screen, and solid values stay on or in the press, to be augered or conveyed to short-term storage on the harvester. Later, these solid values, having high fiber and protein content, may be further processed, for example, for animal feed.

Alternative extractions may also be carried out on-board the harvester. For example, a solvent extraction may be done to solubilize liquid values in order to remove them from solid values. Even mixed solvent extraction, for example, with an emulsion of water and oil, may be carried on on-board.

Then, the liquid values may be subjected to further on-board concentration without heating. Lack of heating is required because many plant liquid values are sensitive to heat. In fact, according to the present invention, as soon as possible after maceration, and in any event immediately upon extraction, the chopped crops, and especially any obtained liquid values, are actively chilled to a temperature substantially less than ambient.

Therefore, further screening, decanting, centrifuging and/or filtration of the liquid values as required on-board the harvester are performed in cooled or chilled equipment. This way, the solids content of liquid #1 is increased, and the solids content of liquid #2 is decreased. In a preferred embodiment wherein the standing crop is green barley, liquid concentrate #1 has high enzyme content, and is suitable for further conventional processing to prepare a water-soluble dietary supplement powder. Also, liquid concentrate #2 has high electrolyte/minerals content and is suitable for further processing as a rinse/back-wash stream in the powdered dietary supplement production discussed above.

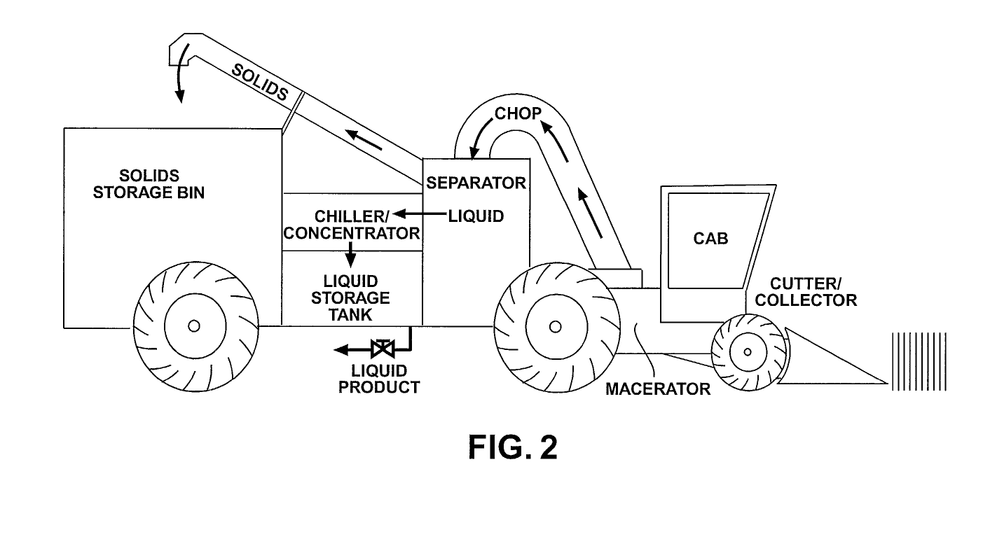

Referring to FIG. 2, there is depicted a schematic side view of one embodiment of the in-field harvester/concentrator of the present invention. The apparatus is preferably a self-propelled cab with a cutter/collector on its front end. A primary engine may drive and move the cab, and a secondary engine may power the macerator, and other auxiliary equipment. The chopped crop is conveyed to the extractor section, which may be part of the cab vehicle, or towed separately on a trailer. The extractor and attendant chiller/concentrator may also be powered by the secondary engine, either through an electricity generator, or hydraulic pump/power-take-off (pto). Refrigeration may begin at the chiller/concentrator section, or may begin earlier, as early as the cutter/collector step in the process, via heat exchange cooling. In any event, the temperature of the liquid values is maintained on-board the harvester at less than about 20° C., and preferably, as low as 5° C.

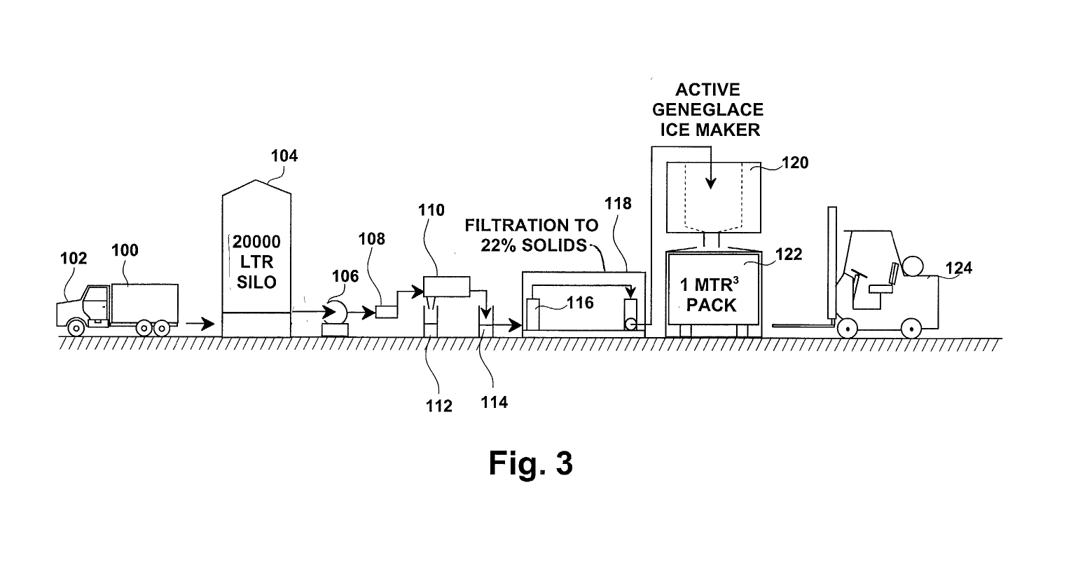

Referring to FIG. 3, there is depicted further processing of chilled juice 100 coming in from the field in a tanker truck 102. Preferably, if the chilled juice 102 is from green barley plants, after some field concentration and cooling it is about 8.5% total solids, and at about 20° C. or less, preferably at about 5° C.

The chilled juice 102 is off-loaded from truck 102, and stored temporarily in cooled and/or insulated silo 104. This way, during high-volume production time, like during harvest, for example, many tank loads from trucks may be off-loaded and stored in silo 104, which may be a plurality of individual silos. Also, this way further down-stream processing may be performed in a more steady state manner, with the volume of silo 104 being used to even out the in-flow from off-loading trucks, and the out-flow to further down-stream processing.

From silo 104, charge pump 106 takes the stored, chilled juice, and sends it on its way for further down-stream processing. For example, the chilled juice will preferably pass through inline filter 108, which may contain successively micro and nano-filtration modules. After filtration, the chilled juice will preferably pass through decanter 110 which will further separate the filtered juice into a first lower solids fraction, gathered in first tank 112, and a second higher solids fraction, gathered in second tank 114. From second tank 114, the second higher solids fraction is supplied to high-pressure feed pump 116 and passed through a reverse-osmosis (R/O) unit 118. The retentate product from R/O unit 118, at this point containing about 22% total solids, is further passed, for example, to ice maker 120 where it may be frozen. If frozen, the cold and concentrated juice may be formed into large bricks 122, and moved by forklift 124 to be stored in an ice warehouse (not shown). This freezing alternative permits great flexibility in the processing of the juice extract. For example, during high-volume production times like harvest, the frozen bricks may be stored until further down-stream processing is available.

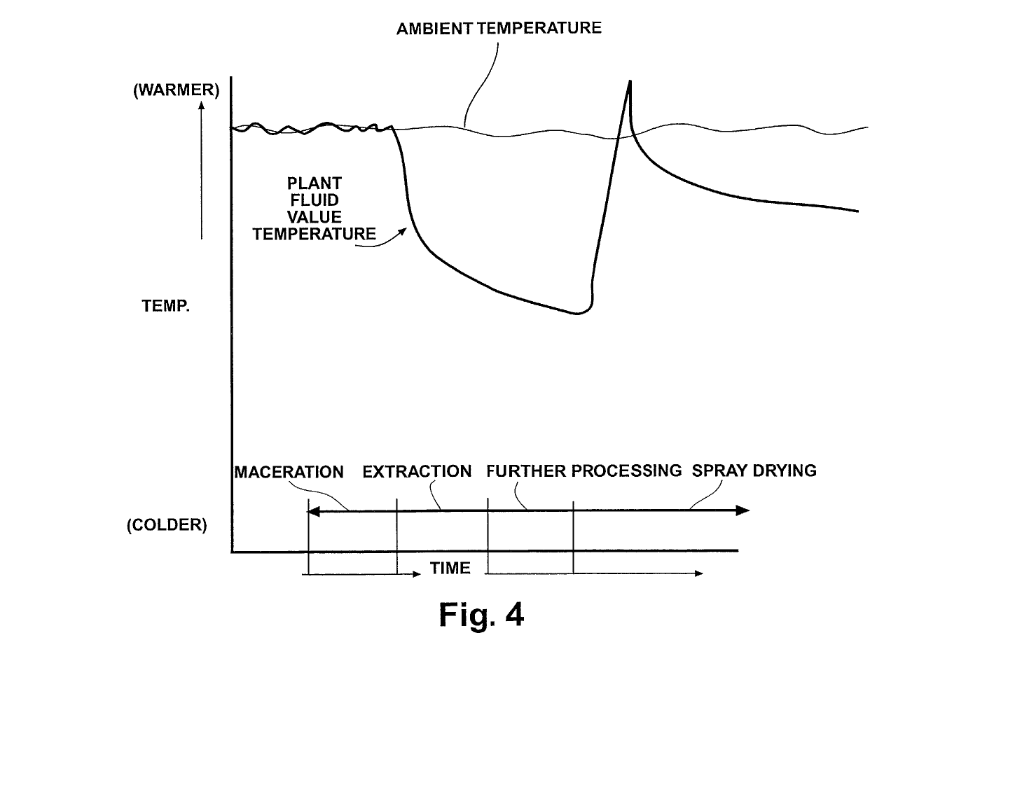

Referring to FIG. 4, there is depicted a schematic temperature time chart for an embodiment of the process of the invention. According to FIG. 4, immediately upon extraction the obtained liquid values are actively cooled to substantially less than ambient temperature. Preferably, the liquid values are cooled to less than about 20° C., and preferably, as low as 5° C. This reduced temperature of the cold juice is continuously maintained during further processing. “Further processing” means increasing the total solids concentration of the chilled liquid values.

Although this invention has been described above with reference to particular means, materials and embodiments, it is to be understood that the invention is not limited to these disclosed particulars, but extends instead to all equivalents within the scope of the following claims.

I claim:

1. A method for concentrating a liquid plant extract where the extract is actively cooled and continuously maintained at a temperature less than ambient during concentration of the extract in order to increase its total solids.

2. The method of claim 1 wherein the liquid plant extract is immediately actively cooled upon extraction.

3. The method of claim 1, wherein the liquid plant extract is cooled to less than about 20 degrees C., and preferably, as low as 5 degrees C.

4. The method of claim 1, wherein said actively cooling comprises refrigerating.
